(19) United States
(12) Patent Application Publication
(10) Pub. No.: US 2003/0144588 A1
(43) Pub. Date: Jul. 31, 2003

(54) Insertable fat suppression enhancer for use with magnetic resonance imaging

(76) Inventors: Eva Zinreich, Owings Mills, MD (US); Helen Zinreich Shafer, Owings Mills, MD (US)

Correspondence Address: HAHN LOESER & PARKS, LLP, TWIN OAKS ESTATE, 1225 W. MARKET STREET, AKRON, OH, 44313, US

(21) Appl. No.: 10/061,868

(22) Filed: Jan. 31, 2002

(51) Int. Cl. A61B 5/05

(52) U.S. Cl. 600/410

(57) ABSTRACT

An insertable enhancer for improving the clarity and resolution of magnetic resonance images, which is constructed from plasticized polyvinyl chloride. The enhancer is adapted for placement into orifices or canals of the body, such as the ear canal, nasal passages, G.I tract or other cavities.

### FIELD OF THE INVENTION

The present invention relates generally to improving the clarity and resolution of images taken during magnetic resonance imaging. More specifically, the present invention relates to a tissue equivalent plastic which is adapted for insertion into canals or orifices of a patient's body and improves the clarity and resolution of images taken of anatomical regions which have excess fat content, while also improving the image of non-fatty tissues.

### BACKGROUND OF THE INVENTION

Magnetic resonance imaging, (“MRI”) is a method by which the location, size, and conformation of organs and other structures of the body can be ascertained.

In the typical MRI system, a magnetic field is established across a body to align the spin axes of the nuclei of a particular chemical element, usually hydrogen, with the direction of the magnetic field. The aligned, spinning nuclei execute precessional motions around the aligning direction of the magnetic field. For the aligned, spinning nuclei, the frequency at which they precess around the direction of the magnetic field is a function of the particular nucleus which is involved and the magnetic field strength. The selectivity of this precessional frequency with respect to the strength of the applied magnetic field is very short and this precessional frequency is considered a resonant frequency.

In an ordinary MRI system, after the nuclei have been aligned or polarized, a burst of radio frequency energy at the resonant frequency is radiated at the body of the patient to produce a coherent deflection of the spin alignment of the selected nuclei. When the deflecting radio energy is terminated, the deflected or disturbed spin axes are reoriented or realigned, and in this process radiate a characteristic radio frequency signal which can be detected by an external coil and then analyzed by the MRI system to establish image contrast between different types of tissues in the body.

Two parameters are used to measure the response of the magnetized sample to a disturbance of its magnetic enviromnent. One is T1 or longitudinal relaxation time, the time it takes the sample to become magnetized or polarized after being placed in a external magnetic field; the other is T2, the spin relaxation time, a measure of the time the sample holds a temporary transverse magnetization which is perpendicular to the external magnetic field. Images based on proton density can be modified by these two additional parameters to enhance differences between tissues.

Hydrogen is usually selected as the basis for MRI scanning because of its prominent magnetic qualities. Hydrogen is easily polarized as it has only a single proton nucleus. Further, hydrogen is abundant in water, a major component of the human body. Tissues which have a high content of water, and thus hydrogen and hydrogen protons are deemed “protonated” and provide strong images during MRI. A disadvantage to hydrogen scanning, however, is that water is a major component of most of the tissues and organs of the body and therefore, most of the tissues of the body are imaged by the MRI system, making it difficult to distinguish between the various tissues with similar hydrogen content during MRI scanning.

When scanning for hydrogen atoms, the images formed in magnetic resonance imaging are really a converted visual display of the otherwise invisible radio waves emitted by protons which are detected by the MRI receiving coil. During this process, tissue areas which have no hydrogen atoms emit no radio waves, and thus the MR image of this tissue is black. Tissues which have a high hydrogen content, on the other hand, may emit a large amount of radio waves depending on the scanning criteria. These signals are converted into a correspondingly bright visual display image. Normally grey scale assignment, based upon the relative energy or signal intensities received from the tissues, is utilized in order that the user may more easily distinguish the various tissues and organs imaged. On these grey scale images, low or no signal is designated as black, and very high signals are assigned a lighter shade of grey or white.

Occasionally, abundant tissues which create a bright signal may overwhelm the signal generated by tissues which are less abundant and have different hydrogen, and thus different proton contents. This may visually mask the image produced by the latter tissue and obscure a disease process or anatomy. Furthermore, the skin air interface can cause unclear or indiscernible images. The skin air interface creates problems during imaging based upon the differences in magnetic characteristics of the anatomical region being imaged and the air surrounding the patients body.

Various methods have been used in trying to separate the signals coming from the various tissues of the body and thereby produce more distinct images. One such method involves nullifying the signal received from a certain tissue. This is done by utilizing spin echo and gradient echo presaturation pulse sequences based upon information about subtle differences in the precessional frequency of hydrogen atoms as they associate with fatty versus non-fatty tissues. For example, in order to improve the noticeability of non-fatty tissues which lie in a background of a fatty tissue, the entire tissue is first subjected to a chemically specific saturation radio pulse. This preparatory pulse essentially affects the hydrogen atoms associated with the fat molecules. These pretreated hydrogen atoms have the effect of being briefly deactivated and do not emit a useful signal when the actual imaging portion of the pulse sequence commences. The MR image is then created with little or no signal generated from the fatty tissues. The resultant image will show the non-fatty tissue against a dark background. This process is called chemically selective presaturation of fat, or fat saturation.

This fat saturation process is unreliable however. Because the precessional differences between the fatty and non-fatty tissues are very minute, this technique must be conducted very precisely or non-fatty tissues are inadvertently variably saturated themselves. This problem is further compounded by the fact that the local magnetic environment of tissues changes based upon their position relative to the coil; position in the magnetic bore; and position with respect to organs or tissues with different magnetic susceptibilities such as tissue next to bone, or tissue next to air. Not only is the immediate magnetic environment important, but the actual geometry of the organ or body part plays a major role in determining the fatty tissue's likelihood of being nullified with the fat saturation technique. For example, fat is more likely to be saturated in the rather cylindrical thigh than in the right angle of the ankle.

Further, interpretive problems can arise in several ways. First, if the fat is not saturated effectively, then pathology can be obscured. Second, if the fat is saturated in only portions of the body part being imaged, then the areas not saturated may be misinterpreted as pathologic tissue. Third, drastic alterations in geometry and magnetic susceptibility which naturally occur in the neck, shoulders, ankles and other superficial joints, for example, can lead to inappropriate saturation of non-fatty tissues which are the subject of the examination.

One method occasionally used to improve fat saturation by addressing the above stated limitation of this technique involves placing water bags around the body part being scanned. This technique is useful in that there is improvement in the quality and reliability of the fat saturation technique. This is based on reducing or eliminating the skin-air interface and by effectively changing the perceived geometry of the part of the body being imaged, such as changing the right angle configuration of the ankle to a more favorable cylindrical shape. However, these do not conform to the surface of the body part being imaged.

Further, as water is highly protonated, it creates a correspondingly bright signal surrounding the fatty tissue image. The bright background is a serious disadvantage for this procedure because it is distracting and counteracts the improved visualization produced by using water-filled bags with fat saturation sequences. The bright background is also problematic when adjusting the potentiometers to “window” an image and try to highlight a structure significantly less bright than the adjacent fat. Without the enhancer of the present invention, when “windowing” for these less bright structures, these images will become obscure and lose definition.

Another method that has been developed to improve fat saturation during magnetic resonance imaging is the placing of a fat saturation material such as a fluorocarbon on or around the body part of the patient being examined, eliminating the skin air interface and reducing anatomical imaging problems. This is accomplished by placing a bag containing the fat saturation enhancing material around the body part to be imaged. However, disadvantages such as the necessity to provide bags of different sizes and shapes in order to be placed around the body part to be imaged is inherent in this method. Furthermore, many of these methods do not allow for inserting or implanting these image enhancement bags within canals or orifices of the patient's body such as the patient's ear canal, for example.

Thus, there is a need to provide a product for clarifying images of areas of the anatomy which contain either limited or excess amounts of fat during magnetic resonance imaging, which is capable of being inserted into orifices or canals of the body.

### SUMMARY OF THE INVENTION

In view of the foregoing, it is an object of the present invention to provide an enhancer for improving images of anatomical areas being imaged by MRI.

It is another object of the present invention to provide an enhancer for improving the image of anatomical areas containing excess fat cells adjacent or within the tissue or anatomy of the area being imaged by MRI.

It is a further object of the present invention to provide an enhancer for clarifying images of areas with excess fat content, which is capable of being inserted into an orifice or canal of the patient being imaged.

It is still another object of the present invention to provide an insertable enhancer for clarifying images of areas with excess fat content that is easily used by radiologists and radiology technicians during MRI imaging.

It is yet another object of the present invention to provide an insertable enhancer for clarifying images of areas with excess fat content being imaged by MRI that is relatively inexpensive to produce.

In order to achieve these objects, an enhancer consisting of a material that has a specific gravity within the range of the specific gravity of the tissue of the body being imaged is utilized. This tissue equivalent plastic is placed between the receiving coil of the magnetic resonance imaging system and the patient and/or tissue being examined. This includes placement of this enhancer within an orifice or canal of the patient's body. The enhancing material is shaped to be capable of insertion into a canal or orifice of the patient's body, such as the ear canal, for example. The enhancer is manufactured from plasticized or highly plasticized polyvinyl chloride having a thickness in the range of 0.5 to 4.0 cm.

These and other objects of the present invention will become more readily apparent from a reading of the detailed description taken in conjunction with the drawings.

### BRIEF DESCRIPTION OF THE DRAWINGS

FIG. 1 is a side view of a preferred embodiment of the present invention adapted for insertion into the ear canal.

FIG. 2 is a top view of a preferred embodiment of the present invention adapted for insertion into the ear canal.

### DETAILED DESCRIPTION OF THE PREFERRED EMBODIMENT

Reference will now be made in detail to the present preferred embodiments of the present invention, examples of which are illustrated in the accompanying drawings. Whenever possible, the same reference numbers will be used throughout the drawings to refer to the same or like parts.

As fatty tissue is highly protonated, this tissue produces a strong signal when scanned and appears as bright, or often white on the magnetic resonance image. This bright signal can mask less abundant, non-fatty tissues that are within or surrounded by fatty tissues. Many times, this non-fatty tissue is the area being focused upon when examining these tissues during an MRI examination. Because of the bright signal being emanated by the surrounding fatty tissues, the signal being produced by the non-fatty tissue may be obscured by the fatty tissue signal. It is therefore beneficial to suppress the image produced by the fatty tissues in order to better examine the non-fatty tissues that are the focus of the examination.

Areas with a limited amount of fat content are also difficult to image effectively if surrounded by fat because of an overshadowing effect which is difficult to “window” with width and level potentiometers. Furthermore, imaging anatomical areas having significant external surface areas is problematic due to the skin air interface. The skin air interface causes problems during MRI imaging due to the fact that the magnetic characteristics of the body differ from the magnetic characteristics of the atmosphere surrounding the body. The magnitude of the magnetization is greatly changed in the boundary between the body and air layer. Therefore, the static field is disturbed in the areas surrounding this boundary area. Placing the enhancer of the present invention on or about the patient's anatomy, in between the patient and/or tissue being examined and the receiving coils of the MRI, stabilizes the static magnetic field surrounding the body of the patient.

The present invention acts as an enhancer to facilitate removal of images generated by fatty tissues during MRI, while also decreasing the skin air interface and problems associated therewith. The enhancer is conformed to be capable of insertion into an orifice or canal of the body, such as the ear canal, G.I. tract, urethra, or vagina, for example. This enhancer is composed of plasticized polyvinyl chloride (“PVC”) having a thickness in the range of 0.5 to 4.0 centimeters. Other materials having similar properties, including a specific gravity of approximately 1.0, while also being pliable may also be utilized. An example of a composition from which the insertable enhancer of the present invention may be manufactured, is produced by Dennis Chemical Company under the product name Clear Hot Melt (soft), product number PX-7514-D. The Dennis Chemical Company product number PX-7514-D is a plasticized polyvinyl chloride containing dispersion grade polyvinyl chloride resin (30%-40%), plasticizer for liquid polyvinyl chloride dispersion (30%-40%), and zinc and zinc compound heat stabilizer. The enhancer may also include tint dispersant, containing two pigmented dispersion color additives: ultramarine blue and R/S phthalocyanin blue. The enhancer material can be used in different manners in order to achieve the desired result of clarification of the images of the anatomy having excess fat content. It is important that the material is flexible in order to conform to the surface of the area being imaged to reduce the skin-air interface.

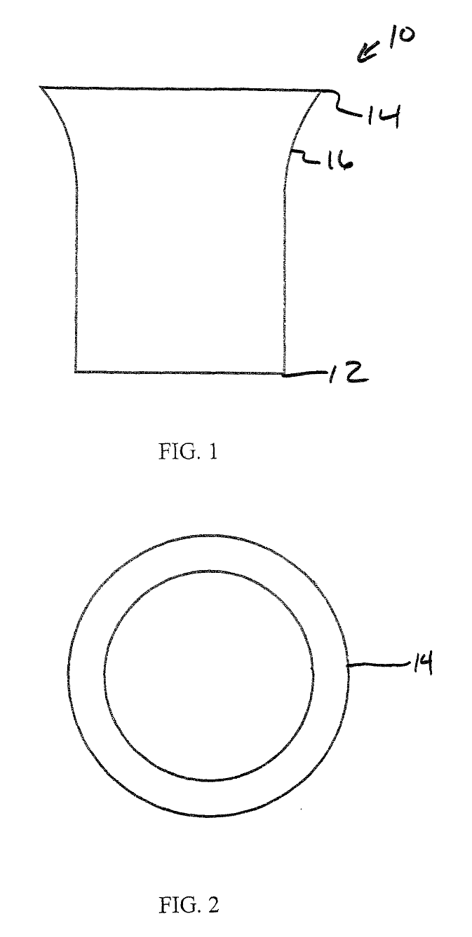

Shown in FIGS. 1 and 2 is a preferred embodiment of the present invention formed for insertion into the ear canal of the patient, shown generally at 10. Imaging the ear canal of a human patient with MRI is problematic due to the amount of fat contained in this region, combined with the effects of the skin air interface. The enhancer of the present invention clarifies the MRI images of the ear canal by suppressing the images created by the fat surrounding this region, while also reducing the skin air interface within the canal itself.

Ear plug 10 has a first end 12 which is adapted for insertion into the patient's ear canal, and a second end 14 which extends outwardly from the patient's ear. Ear plug 10 flares radially outwardly near second end 14 to prevent plug 10 from being inserted too far into the ear canal, and to aid in insertion and removal of plug 10 from the ear canal. Ear plug 10 is formed without any hollow portions or voids, and is solid plasticized polyvinyl chloride. The plasticized PVC conforms to the contours of the ear canal. The required thickness of the enhancer of the present invention varies from patient to patient, and is different for each part of the anatomy being imaged. In a preferred embodiment of an ear plug for use in imaging the ear, the thickness of the enhancing material at first end 12 is approximately 0.5 cm, and the thickness of second end 14 is approximately 1.0 cm. Thus, the diameter of first end 12 is approximately 0.5 cm, and the diameter of second end 14 is approximately 1.0 cm, in this preferred embodiment.

Although the present invention is illustrated for use within the ear canal of a patient, it should be anticipated that this invention can be utilized within various orifices, canals or body cavities, such as the nasal passages, G.I. tract, mouth, urethra, or vagina, for example.

Although the principles, preferred embodiments and preferred operation of the present invention have been described in detail herein, this is not to be construed as being limited to the particular illustrative forms disclosed. They will thus become apparent to those skilled in the art that various modifications of the preferred embodiments herein can be made without departing from the spirit or scope of the invention as defined by the appended claims.

1. An apparatus for improving the clarity and resolution of anatomical images taken during magnetic resonance imaging, said apparatus comprised of plasticized polyvinyl chloride formed in a predetermined shape for insertion into a canal or orifice of the body.

2. An apparatus as recited in claim 1, wherein said plasticized polyvinyl chloride is between 0.5 and 4.0 centimeters thick.

3. An apparatus for improving the clarity and resolution of magnetic resonance images taken of a patient s ear canal, said apparatus comprised of plasticized polyvinyl chloride, and said apparatus being formed as an ear plug for insertion into an ear canal of a patient.

4. An apparatus as recited in claim 3, wherein said ear plug is solid.

5. An apparatus as recited in claim 3, wherein said ear plug comprises a first end, and a second end, wherein said second end has a larger diameter than said first end.

6. An apparatus as recited in claim 5, wherein the diameter of said first end is approximately 0.5 centimeters.

7. An apparatus as recited in claim 5, wherein the diameter of said second end is approximately 1.0 centimeters.
